(19) United States
(12) Patent Application Publication
(10) Pub. No.: US 2012/0225020 A1
(43) Pub. Date: Sep. 6, 2012

(54) UNSATURATED CHOLINE ANALOGS AND CHEMICAL SYNTHESIS THEREOF

(76) Inventors: Eduard Y. Chekmenev, Brentwood, TN (US); Roman V. Shchepin, Nashville, TN (US)

(21) Appl. No.: 13/405,074

(22) Filed: Feb. 24, 2012

Related U.S. Application Data
(60) Provisional application No. 61/446,391, filed on Feb. 24, 2011.

(51) Int. Cl. A61K 49/06; C07F 9/113; C07F 7/04; C07F 7/02; C07F 7/18; C07C 219/20; C07C 67/00

(52) U.S. Cl. 424/9.3; 560/253; 558/166; 560/238; 556/413; 556/410; 556/417; 558/113; 558/90; 558/108

(57) ABSTRACT

This disclosure provides choline analogs comprising the following structure:

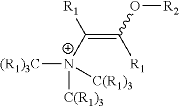

where each R₁ independently is H or isotopically enriched D, R₂ is a protecting group, and N is ¹⁴N or isotopically enriched ¹⁵N. This disclosure also provides methods of making choline analogs, which include performing a protection step on a betaine aldehyde to form a choline analog, and methods of using choline analogs to form hyperpolarized compounds.

### CROSS-REFERENCE TO RELATED APPLICATIONS

This patent application claims priority to U.S. Provisional Patent Application No. 61/446,391 filed Feb. 24, 2011, the entire content of which is incorporated herein by reference.

### STATEMENT REGARDING FEDERAL FUNDING

This invention was made with government support under National Institutes of Health grant numbers 1R00CA134749 and 3R00CA134749-02S1. The government has certain rights in the invention.

### INTRODUCTION

Hyperpolarized MRI offers a sensitivity increase by 4-6 orders of magnitude as compared to conventional MRI. Hyperpolarized MRI contrast agents are similar to PET tracers in that they enable imaging of components (e.g. metabolites) of specific biochemical pathways, and observation of changes in the concentrations of these components. Hyperpolarized MRI thus may be used to identify aberrant metabolism that may indicate the presence of cancer cells. For example, MRI using a hyperpolarized ¹³C-pyruvate contrast agent has been shown to be useful for diagnosing cancer and monitoring responses to cancer treatment, because pyruvate is a key metabolite in glycolysis, which is elevated in cancer cells. There are several key advantages to hyperpolarized MRI. These advantages may include that the hyperpolarized contrast agents: (i) are non-radioactive; (ii) allow for sub-second imaging speed; (iii) exhibit a fast metabolic uptake, which translates to short waiting time; and (iv) are rapidly cleared by the body, thereby permitting same-day follow-up scan(s). Hyperpolarized MRI contrast agents may be selected to have relatively low toxicity, and in many cases have already been approved by regulatory agencies for use in their non-hyperpolarized form. The list of known hyperpolarized metabolic contrast agents is rapidly growing, and includes ¹³C-pyruvate, ¹⁵N-choline, ¹³C-glutamine, ¹³C-bicarbonate, etc.

While the lifetime of hyperpolarized contrast agents is significantly shorter than that of PET tracers, it is sufficiently long for administration to a patient, metabolic uptake by cells and tissues, and metabolic turnover. For example, ¹³C and ¹⁵N sites of the above hyperpolarized metabolic contrast agents have somewhat longer in vivo lifetimes than protons, with reported decay constants (T1) reaching about 40 s and about 120 s, respectively. When translated to clinical practice, hyperpolarized MRI is likely to become a game-changing technology that can dramatically accelerate clinical trials and drug discovery, and will increase the diagnosis and treatment of diseases such as cancer. These benefits are due to fundamental properties of hyperpolarized contrast agents that allow for at least one of sub-second imaging time, multiple same-day imaging exams and the potential and practical ability to predict treatment outcome within a day (or even faster in the case of targeted therapies) after initial treatment.

### SUMMARY

The present disclosure provides compositions comprising a compound having the following structure:

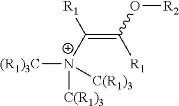

where each R₁ is independently H or isotopically enriched D;

where R₂ is

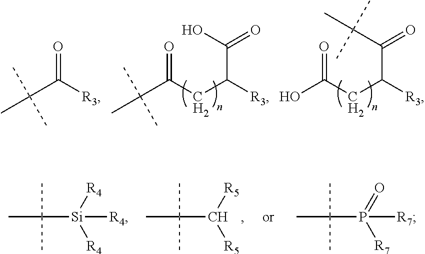

where R₃ is H, an alkyl (e.g., is —CH₃), a cycloalkyl, an alkenyl, a cycloalkenyl, an alkynyl, a cycloalkynyl, an aryl, an alkoxyl, a cyano, a thiol, an amino, or a solid support;

where each R₄ is independently H, a leaving group, an acyl, an alkyl, a cycloalkyl, an alkenyl, a cycloalkenyl, an alkynyl, a cycloalkynyl, an aryl, an alkoxyl, a cyano, a thiol, an amino, or a solid support;

where each R₅ is independently H, a leaving group, an acyl, an alkyl, a cycloalkyl, an alkenyl, a cycloalkenyl, an alkynyl, a cycloalkynyl, an aryl, an alkoxyl, a cyano, a thiol, an amino, or a solid support;

where each R₇ is independently —OH, or an alkoxyl;

where N is ¹⁴N or isotopically enriched ¹⁵N;

where n is an integer from 1-5; and

where at least one of R₂, R₃, R₄, R₅, or R₇ comprises a negatively charged moiety, or the analog further comprises an anion Z^{⊖} (e.g., a halide).

Some compounds, including but not limited to the following, are in the form of salts, such as a chloride or bromide salts:

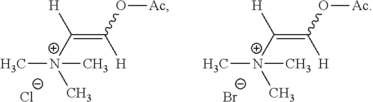

Some compounds include one or more istopically enriched atoms. For example, N may be isotopically enriched ¹⁵N and/or at least one of the R₁ atoms may be isotopically enriched D.

The present disclosure also provides methods of making choline analogs. These methods include performing a protection step on a betaine aldehyde to form the unstaurated choline analogs. Some methods also include synthesizing allyl trimethylammonium from a trimethylammonium and an allyl, and ozonizing the allyl trimethylammonium to form the betaine aldehyde.

In some methods, one or more of the starting materials for synthesizing the choline analog include isotopically enriched atoms. For example, the nitrogen atom of the trimethylammonium may be isotopically enriched ¹⁵N, at least one of the hydrogen atoms of the trimethylammonium may be istopically enriched D, and/or at least one of the hydrogen atoms of the allyl may be isotopically enriched D.

Various protecting steps may be used to form the choline analogs. For example, the protection step may include reacting the betaine aldehyde with any of a wide variety of reactants, including an anhydride, a protecting group that includes silicon, or a phosphine oxide, among others.

The choline analogs of the present disclosure may be used in methods of performing MRI. Specifically, the choline analogs may be reduced with parahydrogen to form a hyperpolarized choline analog, such as hyperpolarized phosphocholine or a hyperpolarized protected choline precursor that may be deprotected to form hyperpolarized choline.

### BRIEF DESCRIPTION OF THE DRAWINGS

FIG. 1 is a flow chart showing the general reaction scheme for synthesizing choline analogs, according to aspects of the present disclosure.

FIG. 2 is a flow chart showing an embodiment of the protection step of FIG. 1.

FIG. 3 is a flow chart showing another embodiment of the protection step of FIG. 1

FIG. 4 is a flow chart showing another embodiment of the protection step of FIG. 1.

FIG. 5 is a flow chart showing yet another embodiment of the protection step of FIG. 1.

FIG. 6 is a flow chart showing yet another embodiment of the protection step of FIG. 1.

FIG. 7 is a flow chart showing the general reaction scheme for using the choline analogs of the present disclosure to produce hyperpolarized choline.

FIG. 8 is a flow chart showing an embodiment of the deprotection step of FIG. 6.

FIG. 9 is a flow chart showing another embodiment of the deprotection step of FIG. 6.

FIG. 10 is a flow chart showing another embodiment of the deprotection step of FIG. 6.

FIG. 11 is a flow chart showing another embodiment of the deprotection step of FIG. 6.

FIG. 12 is a flow chart showing the reaction scheme for synthesizing acetylated 1,2-dehydrocholine chloride.

FIG. 13 is a flow chart showing the reaction scheme for synthesizing acetylated 1,2-dehydrocholine bromide.

FIG. 14 is a ¹H NMR spectra of acetylated 1,2-dehydrocholine.

FIG. 15 is a ¹³C NMR spectra of acetylated 1,2-dehydrocholine.

FIG. 16 is a mass spectrum of acetylated 1,2-dehydrocholine.

FIG. 17 is a ¹H NMR spectra of acetylated 1,2-dehydrocholine in DMSO-d₆ (top) and D₂O (bottom).

FIG. 18 is a flow chart showing the reaction scheme for synthesizing acetylated ¹⁵N-1,2-dehydrocholine bromide.

FIG. 19 is a series of ¹H, ¹³C and ¹⁵N spectra of acetylated ¹⁵N-1,2-dehydrocholine in D₂O and DMSO-d₆.

FIG. 20 is a flow chart showing the reaction scheme for synthesizing acetylated perdeuterated ¹⁵N-1,2-dehydrocholine bromide.

### DETAILED DESCRIPTION

The present disclosure is not limited in its application to the specific details of construction, arrangement of components, or method steps set forth herein. The compositions and methods disclosed herein are capable of being made, practiced, used, carried out and/or formed in various ways. The phraseology and terminology used herein is for the purpose of description only and should not be regarded as limiting. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification, and no structures shown in the drawings, should be construed as indicating that any non-claimed element is essential to the practice of the invention. The use herein of the terms “including,” “comprising,” or “having,” and variations thereof, is meant to encompass the items listed thereafter and equivalents thereof, as well as additional items. Unless specified or limited otherwise, the terms “mounted,” “connected,” “supported,” and “coupled” and variations thereof encompass both direct and indirect mountings, connections, supports, and couplings. Further, “connected” and “coupled” are not restricted to physical or mechanical connections or couplings.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinency of any of the documents cited herein. All references cited herein are fully incorporated by reference, unless explicitly indicated otherwise. The present disclosure shall control in the event there are any disparities.

The following definitions shall apply unless otherwise indicated.

The term “acyl,” as used herein, refers to a group having the general structure —C(═O)R, wherein R is an acyl substituent. Unless otherwise specified, acyl substituents may include, but are not limited to, H, an alkyl, an alkenyl, an alkynyl, a cyclic alkyl, a cyclic alkenyl, and an aryl, among others. Examples of acyl groups include, but are not limited to, —C(═O)CH₃ (acetyl), —C(═O)CH₂CH₃ (propionyl), —C(═O)CH₂CH₂CH₃ (butyryl), and —C(═O)Ph (benzoyl, phenone).

The term “alkenyl,” as used herein, refers to an unsaturated hydrocarbon chain having at least one double bond and 2 to 12 carbon atoms. Suitable examples include alkenyls having 2 to 7 carbon atoms, 2 to 6 carbon atoms, 2 to 5 carbon atoms, 2 to 4 carbon atoms or 2 to 3 carbon atoms. Alkenyl groups may have more than one double bond. Alkenyl groups also may have one or more triple bonds. For example, alkenyl groups may have one or more double bonds and one or more triple bonds. Alkenyl groups may be straight or branched, and branched alkenyl groups may have one or more branches. Alkenyl groups may be unsubstituted or may have one or more independent substituents. Unless otherwise specified, each substituent may include, but is not limited to, an alkyl, a cycloalkyl, a bicycloalkyl, an alkenyl, a cycloalkenyl, a bicycloalkenyl, an alkynyl, an acyl, an aryl, a cyano group, a halogen, a hydroxyl group, a carboxyl group, an isothiocyanoto group, an ether, an ester, a ketone, a sulfoxide, a sulfone, a thioether, a thioester, a thiol group, an amino, an amido, or a nitro group, among others. Each substituent also may include any group that, in conjunction with the alkenyl, forms an ether, an ester, a ketone, a thioether, a thioester, a sulfoxide, a sulfone, an amine or an amide, among others. Some alkenyl groups may have one or more chiral carbons because of the branching or substitution. Chiral alkenyl groups include both (+)dextrorotary and (−)levorotary compounds; “D-” and “L-” chiral compounds, as well as alkenyl groups containing “R-” and “S-” stereocenters. Some alkenyl groups may include one or more heteroatoms.

The terms “alkoxy,” or “alkoxyl,” as used herein, refer to functional groups or substituents having the general structure —OR, where R is an alkoxy substituent. Unless otherwise specified, R is not limited to an alkyl and may include an alkyl, an alkenyl, an alkynyl, a cycloalkyl, a cycloalkynyl, and an aryl, among others.

The term “alkyl,” as used herein, refers to a saturated hydrocarbon chain having 1 to 12 carbon atoms. Suitable examples include alkyls having 1 to 7 carbon atoms, 1 to 6 carbon atoms, 1 to 5 carbon atoms, 1 to 4 carbon atoms or 1 to 3 carbon atoms. Alkyl groups may be straight or branched, and branched alkyl groups may have one or more branches. Alkyl groups may be unsubstituted or may have one or more independent substituents. Unless otherwise specified, each substituent may include, but is not limited to an alkyl, a cycloalkyl, a bicycloalkyl, an alkenyl, a cycloalkenyl, a bicycloalkenyl, an alkynyl, an acyl, an aryl, a cyano group, a halogen, a hydroxyl group, a carboxyl group, an isothiocyanoto group, an ether, an ester, a ketone, a sulfoxide, a sulfone, a thioether, a thioester, a thiol group, an amino, an amido, or a nitro group, among others. Each substituent also may include any group that, in conjunction with the alkyl, forms an ether, an ester, a ketone, a thioether, a thioester, a sulfoxide, a sulfone, an amine or an amide, among others. Some alkyl groups may have one or more chiral carbons because of the branching or substitution. Chiral alkyl groups may include both (+)dextrorotary and (−)levorotary compounds; “D-” and “L-” chiral compounds, as well as alkyl groups containing “R-” and “S-” stereocenters. Some alkyl groups may include one or more heteroatoms.

The term “alkynyl,” as used herein, refers to an unsaturated hydrocarbon chain having at least one triple bond and 2 to 12 carbon atoms. Suitable examples include alkynyls having 2 to 7 carbon atoms, 2 to 6 carbon atoms, 2 to 5 carbon atoms, 2 to 4 carbon atoms or 2 to 3 carbon atoms. Alkynyl groups may have more than one triple bond. Alkynyl groups also may have one or more double bonds. For example, alkynyl groups may have one or more double bonds and one or more triple bonds. Alkynyl groups may be straight or branched, and branched alkynyl groups may have one or more branches. Alkynyl groups may be unsubstituted or may have one or more independent substituents. Unless otherwise specified, each substituent may include, but is not limited to an alkyl, a cycloalkyl, a bicycloalkyl, an alkenyl, a cycloalkenyl, a bicycloalkenyl, an alkynyl, an acyl, an aryl, a cyano group, a halogen, a hydroxyl group, a carboxyl group, an isothiocyanoto group, an ether, an ester, a ketone, a sulfoxide, a sulfone, a thioether, a thioester, a thiol group, an amino, an amido, or a nitro group, among others. Each substituent also may include any group that, in conjunction with the alkynyl, forms an ether, an ester, a ketone, a thioether, a thioester, a sulfoxide, a sulfone, an amine or an amide, among others. Some alkynyl groups may have one or more chiral carbons because of the branching or substitution. Chiral alkynyl groups include both (+)dextrorotary and (−)levorotary compounds; “D-” and “L-” chiral compounds, as well as alkynyl groups containing “R-” and “S-” stereocenters. Some alkynyl groups may include one or more heteroatoms.

The term “anhydride,” as used herein, refers to any compound R¹—O—R², where R¹ and R² each are acyl groups. R¹ and R² may be the same acyl groups, in which case the anhydride may be a symmetrical anhydride. R¹ and R² also may be different acyl groups, in which case the anhydride may be an unsymmetrical anhydride. R¹ and R² also may be covalently attached to one another, in which case the anhydride may be a cyclic anhydride.

The term “anion,” as used herein, refers to a negatively charged atom or group of atoms in a molecule, such as, for example, a halide (e.g., fluoride, chloride, bromide or iodide), N₃⁻, PO₄³⁻, HPO₄²⁻, H₂PO₄²⁻, SO₄²⁻, HSO₄⁻, NO₃⁻, ClO₄⁻, CO₃²⁻, HCO₃⁻, CrO₄²⁻, Cr₂O₇²⁻, CN⁻, OH⁻, Cr₂O₄²⁻, MnO₄⁻, BF₄⁻, B(C₆H₅)₄⁻, PF₆⁻, HCOO⁻, CH₃COO⁻, CF₃COO⁻, CF₃SO₃⁻, PtCl₄²⁻, and the like.

The term “aryl,” as used herein, refers to any functional group or substituent group derived from an aromatic hydrocarbon ring system. Aromatic rings may be monocyclic or fused multicyclic ring systems. Monocyclic aromatic rings may contain from about 4 to about 10 carbon atoms, such as from 5 to 7 carbon atoms, or from 5 to 6 carbon atoms in the ring. Multicyclic aromatic rings may contain from about 4 to about 10 carbon atoms per ring, and from 2 to about 4 rings, where adjacent rings may share two or more carbon atoms. Aromatic rings may be unsubstituted or may have one or more independent substituents on the ring. Unless otherwise specified, each substituent may include, but is not limited to an alkyl, a cycloalkyl, a bicycloalkyl, an alkenyl, a cycloalkenyl, a bicycloalkenyl, an alkynyl, an acyl, an aryl, a cyano group, a halogen, a hydroxyl group, a carboxyl group, an isothiocyanoto group, an ether, an ester, a ketone, a sulfoxide, a sulfone, a thioether, a thioester, a thiol group, an amino, an amido, or a nitro group, among others. Each substituent also may include any group that, in conjunction with the aryl, forms an ether, an ester, a ketone, a thioether, a thioester, a sulfoxide, a sulfone, an amine or an amide, among others. Some aryl groups may include one or more heteroatoms.

The term “amide,” as used herein, refers to a group having the general structure RC(═O)NR¹R², wherein N, R¹ and R² are an amido group and R is an independent substituent that, unless otherwise specified, may include an alkyl, an alkenyl, an alkynyl, a cycloalkyl, a cycloalkenyl, and an aryl among others.

The term “amido,” as used herein, refers to a group having the general structure —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂. as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl. Cyclic amido groups may be substituted on their ring by other substituents and/or may include one or more other heteroatoms.

The term “amine,” as used herein, refers to a group having the general structure NR¹R²R³, wherein N, R¹ and R² are an amino group attached to R³, which is an independent amino substituent, as defined below.

The term “amino,” as used herein, refers to a group having the general structure —NR¹R², wherein R¹ and R² are independently amino substituents. Unless otherwise specified, amino substituents may include, but are not limited to, hydrogen, an alkyl, an alkenyl, an alkynyl, a cycloalkyl, a cycloalkenyl, or an aryl group, among others or, in the case of a “cyclic” amino group, R¹ and R², taken together with the nitrogen atom to which they are attached form a heterocyclic ring having from 3 to 8 ring atoms. Examples of amino groups include, but are not limited to, —NH₂, —NHCH₃, —NHCH(CH₃)₂, —N(CH₃)₂, —N(CH₂CH₃)₂, —NHPh, etc. Examples of cyclic amino groups include, but are not limited to, aziridinyl, azetidinyl, pyrrolidinyl, piperidino, piperazinyl, perhydrodiazepinyl, morpholino, and thiomorpholino. Cyclic amino groups also may be substituted on their ring by other substituents, and/or may include one or more other heteroatoms.

The term “carboxyl,” as used herein, refers to the group —COOR, where R is a carboxyl substituent. Unless otherwise specified, carboxyl substituents may include, but are not limited to an alkyl, an alkenyl, an alkynyl, a cycloalkyl, a cycloalkynyl, and an aryl, among others.

The term “cycloalkenyl,” as used herein, refers to any functional group or substituent having an unsaturated hydrocarbon ring that is non-aromatic. Cycloalkenyls have one or more double bonds. Cycloalkenyls are monocyclic, or are fused, spiro, or bridged bicyclic ring systems, where the term “bicycloalkenyl,” as used herein, refers to such bicyclic unsaturated ring structures. Monocyclic cycloalkenyls contain from about 3 to about 10 carbon atoms, such as from 4 to 7 carbon atoms, or from 5 to 6 carbon atoms in the ring. Bicycloalkenyls contain from 5 to 12 carbon atoms, such as from 8 to 10 carbon atoms in the ring. Cycloalkenyls may be unsubstituted or may have one or more independent substituents on the ring. Unless otherwise specified, each substituent may include, but is not limited to an alkyl, a cycloalkyl, a bicycloalkyl, an alkenyl, a cycloalkenyl, a bicycloalkenyl, an alkynyl, an acyl, an aryl, a cyano group, a halogen, a hydroxyl group, a carboxyl group, an isothiocyanoto group, an ether, an ester, a ketone, a sulfoxide, a sulfone, a thioether, a thioester, a thiol group, an amino, an amido, or a nitro group, among others. Each substituent also may include any group that, in conjunction with the cycloalkenyl, forms an ether, an ester, a ketone, a thioether, a thioester, a sulfoxide, a sulfone, an amine or an amide, among others. Cycloalkenyl groups may have one or more chiral carbons because of the substitution. Some cycloalkenyl groups may include one or more heteroatoms. Examples of cycloalkenyls include, but are not limited to cyclopropenyl, cyclohexenyl, 1,4 cyclooctadienyl, and bicyclooctenyl, among numerous others.

The term “cycloalkyl,” as used herein, refers to any functional group or substituent having a saturated hydrocarbon ring that is non-aromatic. Cycloalkyls are monocyclic, or are fused, spiro, or bridged bicyclic saturated ring systems, where the term “bicycloalkyl,” as used herein, refers to such bicyclic saturated ring structures. Monocyclic cycloalkyls contain from about 3 to about 10 carbon atoms, such as from 4 to 7 carbon atoms, or from 5 to 6 carbon atoms in the ring. Bicycloalkyls contain from 5 to 12 carbon atoms, such as from 8 to 10 carbon atoms in the ring. Cycloalkyls may be unsubstituted or may have one or more independent substituents on the ring. Unless otherwise specified, each substituent may include, but is not limited to an alkyl, a cycloalkyl, a bicycloalkyl, an acyl, an aryl, a cyano group, a halogen, a hydroxyl group, a carboxyl group, an isothiocyanoto group, an ether, an ester, a ketone, a sulfoxide, a sulfone, a thioether, a thioester, a thiol group, an amino, an amido, or a nitro group, among others. Each substituent also may include any group that, in conjunction with the cycloalkyl, forms an ether, an ester, a ketone, a thioether, a thioester, a sulfoxide, a sulfone, an amine or an amide, among others. Cycloalkyl groups may have one or more chiral carbons because of the substitution. Some cycloalkyl groups may include one or more heteroatoms. Examples of cycloalkyls include, but are not limited to cyclopentyl, cyclohexyl, cycloheptyl, cyclooctyl, bicyclooctyl, and tetrahydropyran, among numerous others.

The term “cyano,” as used herein, refers to the group —CN.

The term “ether,” as used herein, refers to the group ROR′, where R and R′ are ether substituents. Unless otherwise specified, R and R′ each may include an alkyl, an alkenyl, an alkynyl, a cycloalkyl, a cycloalkynyl, and an aryl, among others.

The term “ester,” as used herein, refers to the group RC(═O)OR′, wherein R and R′ are ester substituents. Unless otherwise specified, R and R′ each may include an alkyl, an alkenyl, an alkynyl, a cycloalkyl, a cycloalkynyl, and an aryl, among others.

The term “halogen,” as used herein, refers to fluorine, chlorine, bromine or iodine. Halogens are not heteroatoms.

The term “heteroatom,” as used herein, refers to a nitrogen, sulfur, or oxygen atom. Groups containing more than one heteroatom may contain different heteroatoms.

The term “hydroxyl,” as used herein, refers to the group —OH.

The term “isothiocyanoto,” as used herein, refers to the group: —SCN.

The term “isotopically enriched,” as used herein with reference to any particular isotope of any particular atom of a compound, means that in a composition comprising a plurality of molecules of the compound, the amount (e.g., fraction, ration or percentage) of the plurality of molecules having the particular isotope at the particular atom is substantially greater than the natural abundance of the particular isotope, due to synthetic enrichment of the particular atom with the particular isotope. For example, a composition comprising a compound with an isotopically enriched ¹⁵N atom at a particular location includes a plurality of molecules of the compound where, as a result of synthetic enrichment, the percentage of the plurality of molecules having ¹⁵N at that location is greater than about 1% (the natural abundance of ¹⁵N is substantially less than 1%), and in many cases is substantially greater than about 1%. Similarly, a composition comprising a compound with an isotopically enriched deuterium (D) atom at one or more particular locations includes a plurality of molecules of the compound, where as a result of synthetic enrichment, the percentage of the plurality of molecules having D at each of the one or more particular locations is greater than about 1% (the natural abundance of D is substantially less than 1%), and in many cases is substantially greater than about 1%. In some cases, a composition comprising a compound with an isotopically enriched atom at a particular location may include a plurality of molecules of the compound, where the amount of the plurality of molecules having the isotope at the location may be at least about two-or-more-fold greater than the natural abundance of the isotope, including but not limited to at least about two-fold, at least about three-fold, at least about four-fold, at least about five-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, and at least about 200-fold, among others. In some cases, a composition comprising a compound with an isotopically enriched atom at a particular location also may include a plurality of molecules of the compound where, as a result of synthetic enrichment, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, of the plurality of molecules have the isotope at the location.

The term “ketone,” as used herein, refers to the group RC(═O)R′, wherein R and R′ are ketone substituents. Unless otherwise specified, R and R′ each may include an H, an alkyl, an alkenyl, an alkynyl, a cycloalkyl, a cycloalkenyl, and an aryl, among others.

The term “leaving group,” as used herein, refers to any molecular moiety that departs with a pair of electrons in hydrolytic bond cleavage. A leaving group may include, but is not limited to, a halogen (i.e., fluorine, chlorine, bromine, or iodine), a tosyl group (i.e., p-toluenesulfonyl), a methanesulfonyl group (i.e., CH₃SO₂—), a trifluoromethanesolfonyl group (i.e., CF₃SO₂—), and a trifluoroacetate group (i.e., CF₃CO₂—), among others.

The term “natural abundance,” as used herein with reference to any particular isotope of an element, refers to the abundance of the isotope as naturally found on the planet Earth. For example, the natural abundance of ¹⁵N on the planet Earth is generally regarded to be about 0.37% (i.e., substantially less than about 1%), while the natural abundance of deuterium (D) on the planet Earth is generally regarded to be about 0.015% (i.e., substantially less than about 1%).

The term “nitro,” as used herein, refers to the group —NO₂.

The term “saturated,” as used herein, means that a moiety has no units of unsaturation.

The term “solid support,” as used herein, refers to any type of solid support now known or hereinafter devised for attaching or otherwise binding molecules to the surface thereof, including, but not limited to, beads, gels, columns, chips, and microarray wells, among others, formed of glass(es), resin(s), polystyrene(s), polyamide(s), PEG, magnetizable materials, and silica(s), among others. More specific examples of solid supports include, but are not limited to, Dowex® and Amberlyst™ ion exchange resins.

The term “sulfoxide,” as used herein, refers to the group RS(═O)R′, wherein R and R′ are sulfoxide substituents. Unless otherwise specified, R and R′ each may include an alkyl, and alkenyl, an alkynyl, a cycloalkyl, a cycloalkenyl, and an aryl, among others.

The term “sulfone,” as used herein, refers to the group RS(═O)₂R′, wherein R and R′ are sulfone substituents. Unless otherwise specified, R and R′ each may include an alkyl, and alkenyl, an alkynyl, a cycloalkyl, a cycloalkenyl, and an aryl, among others.

The term “thiol,” as used herein, refers to the group —SR, where R is a thio substituent. Unless otherwise specified, R may include an alkyl, an alkenyl, an alkynyl, a cycloalkyl, a cycloalkynyl, and an aryl, among others.

The term “thioether,” as used herein, refers to the group RSR', where R and R′ are thioether substituents. Unless otherwise specified, R and R′ each may include an alkyl, an alkenyl, an alkynyl, a cycloalkyl, a cycloalkynyl, and an aryl, among others.

The term “thioester,” as used herein, refers to the group RC(═O)SR', wherein R and R′ are thioester substituents. Unless otherwise specified, R and R′ each may include an alkyl, an alkenyl, an alkynyl, a cycloalkyl, a cycloalkynyl, and an aryl, among others.

The term “unsaturated,” as used herein, means that a moiety has one or more carbon-carbon double or triple bonds.

While hyperpolarized metabolic imaging holds great promise for detecting and imaging cancer and other diseases, and for monitoring the response to a particular treatment, its clinical implementation hinges on the ability to produce short-lived contrast hyperpolarized contrast agents on site. While hyperpolarized ¹³C-pyruvate (produced by Dynamic Nuclear Polarization (DNP) technology) is the most well-characterized hyperpolarized contrast agent, hyperpolarized ¹⁵N-choline (produced by DNP) has been shown to be an effective hyperpolarized contrast agent for in vivo reporting on metabolic imbalances related to choline metabolism, which is indicative of the elevated cell membrane synthesis or proliferation commonly associated with cancer. Unfortunately, hyperpolarization of ¹⁵N-choline by DNP takes several hours and results in a material with several fold less hyperpolarization than the ¹³C-pyruvate produced by DNP.

Parahydrogen Induced Polarization (PHIP) is another method of hyperpolarization, and is extremely fast relative to DNP. PHIP has been used to produce hyperpolarized ¹³C-succinate (Polarization P=20% and above) in less than 1 minute. Prior to the present invention, hyperpolarized ¹⁵N-choline has not been produced with PHIP.

The present disclosure provides choline analogs having the following structure:

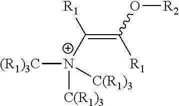

where each R₁ independently is H or is isotopically enriched D;

where R₂ is

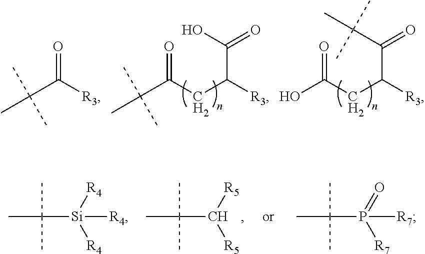

where each R₃ independently is H, an alkyl (e.g., is —CH₃), a cycloalkyl, an alkenyl, a cycloalkenyl, an alkynyl, a cycloalkynyl, an aryl, an alkoxyl, a cyano, a thiol, an amino, or a solid support;

where R₃ suitably is H, an alkyl (e.g., is —CH₃), a cycloalkyl, an alkenyl, a cycloalkenyl, an alkynyl, a cycloalkynyl, an aryl, or a solid support

where each R₄ independently is H, a leaving group, an acyl, an alkyl, a cycloalkyl, an alkenyl, a cycloalkenyl, an alkynyl, a cycloalkynyl, an aryl, an alkoxyl, a cyano, a thiol, an amino, or a solid support;

where each R₅ independently is H, a leaving group, an acyl, an alkyl, a cycloalkyl, an alkenyl, a cycloalkenyl, an alkynyl, a cycloalkynyl, an aryl, an alkoxyl, a cyano, a thiol, an amino, or a solid support;

where each R₇ independently is —OH, or an alkoxyl;

where N is ¹⁴N or isotopically enriched ¹⁵N;

where n is an integer from 1-5; and

where at least one of R₂, R₃, R₄, R₅ or R₇ comprises a negatively charged moiety,

or the analog further comprises an anion Z^{⊖} (e.g., a halide).

In some embodiments, the choline analogs have the following structure:

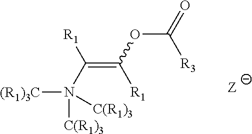

where each R₁ independently is H or is isotopically enriched D;

where R₃ is H, an alkyl (e.g., is —CH₃), a cycloalkyl, an alkenyl, a cycloalkenyl, an alkynyl, a cycloalkynyl, an aryl, or a solid support;

where N is ¹⁴N or isotopically enriched ¹⁵N; and

Z^{⊖} is an anion (e.g., a halide).

In some embodiments, at least one R₁ is isotopically enriched D. In some embodiments, R₃ is alkyl (e.g., —CH₃). In some embodiments, Ze is a halide, e.g., chloro or bromo.

In some embodiments, the choline analogs have the following structure:

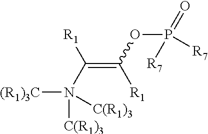

where each R₁ is independently H or is isotopically enriched D;

where each R₇ is independently —OH, or an alkoxyl;

where N is ¹⁴N or isotopically enriched ¹⁵N; and

where at least one R₇ comprises a negatively charged moiety (e.g., is a deprotonated —OH), or the analog further comprises an anion Z^{⊖} (e.g., a halide).

Some choline analogs are in the form of salts, such as a chloride or bromide salts. As discussed in more detail below, the choline analogs of the present disclosure may be used as precursors to MRI contrast agents. For example, as discussed in more detail below, the choline analogs may be reduced with parahydrogen to form hyperpolarized saturated choline analogs that can be used as MRI contrast agents, or that can be further modified to form hyperpolarized choline for use as an MRI contrast agent. Because MRI contrast agents are administered to humans and, as a general rule, chloride may be more safetly administered to humans than bromine, choline analogs intended for use as MRI contrast agents preferably may be chloride salts.

Some unstaurated choline analogs include one or more istopically enriched atoms. For example, N may be isotopically enriched ¹⁵N and/or R₁ may be isotopically enriched D. The choline analogs of the present disclosure preferably may be istopically enriched with ¹⁵N so that substantially more hyperpolarized saturated choline analog is formed by the reaction with parahydrogen, and the hyperpolarized saturated choline analog can be used effectively as an MRI contrast agent or as a precursor to an MRI contrast agent. The choline analogs also preferably may include one or more R₁ atomsisotopically enriched with D, because deuteration may improve ¹⁵N polarization efficiency during PHIP by >20% and may extend the lifetime of the contrast agent. For example, the lifetime of tracer (T1) for 1-¹³C-succinate-D2,3 (formed by PHIP of deuterated fumarate) was more than 300% longer than the lifetime of tracer (T1) for 1-¹³C-succinate formed from (PHIP of non-isotopically enriched fumarate). See Chekmenev, E. Y.; Hoevener, J. B.; Norton, V. A.; Harris, K. C.; Batchelder, L. S.; Bhattacharya, P.; Ross, B. D. Weitekamp, D. P. “PASADENA Hyperpolarization of Succinic Acid for MRI and NMR” J. Am. Chem. Soc. 2008, 130, 4212-4213, the complete disclosure of which is herein incorporated by reference for all purposes. Moreover, deuteration of the protons on the methylene moieties associated with the choline analogs (as compared to the nine methyl protons associated with trimethylammonium moiety) is more important for polarization transfer using PHIP.

As discussed above, R₂ may be a variety of different chemical moieties, including, but not limited to:

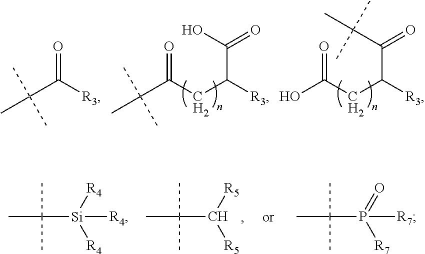

R₂ also may be referred to as a protecting group. The protecting group may be identical to the corresponding moiety of a naturally-occurring metabolite of choline. For example, the protecting group may be an acetyl or phosphite moiety, among others, such that reduction of the unsaturated choline analog with parahydrogen forms a hyperpolarized form of the naturally-occurring acetylcholine or phosphocholine, respectively. In such cases, the hyperpolarized form of the naturally-occurring choline metabolite may be directly administered for use as an MRI contrast agent. The protecting group also may be non-analogous to any corresponding moiety of a naturally-occurring metabolite of choline, such that reduction of the unsaturated choline analog with parahydrogen forms a hyperpolarized non-naturally occurring choline analog. Some of these hyperpolarized non-naturally occurring choline analogs may be useful as contrast agents, and also may be non-toxic to humans, in which case the hyperpolarized non-naturally occurring choline analog may be directly administered to humans. Some of the hyperpolarized non-naturally occurring choline analogs also may be further chemically modified to form a compound useful as a contrast agent administerable to humans. For example, as discussed in more detail below, some hyperpolarized non-naturally occurring choline analogs may be deprotected (such as with treatment with acids or bases) to hydrolyze the protecting group and form hyperpolarized choline for use as an MRI contrast agent.

Various factors may affect whether a specific protecting group is selected. The protecting group may be selected, in part, to stabilize the unsaturated choline analog by inhibiting reduction of the C═C bond prior to the desired use of the unsaturated choline analog. For example, as discussed in the Examples below, acetyl 1,2-dehydrocholine has been shown to be stable for several hours in aqueous medium. In some cases, protecting groups may improve the stability of the corresponding contrast agent in vivo, and/or may provide contrast agents having more favorable properties (such as pharmokinetics, binding affinity, toxicity, and like) for analyzing a particular disease or metabolic disorder. Some protecting groups may be selected based on how easy they are to synthesize and/or use in PHIP. As disclosed in more detail below, choline analogs having the various protecting groups shown above may be synthesized by a variety of different methods.

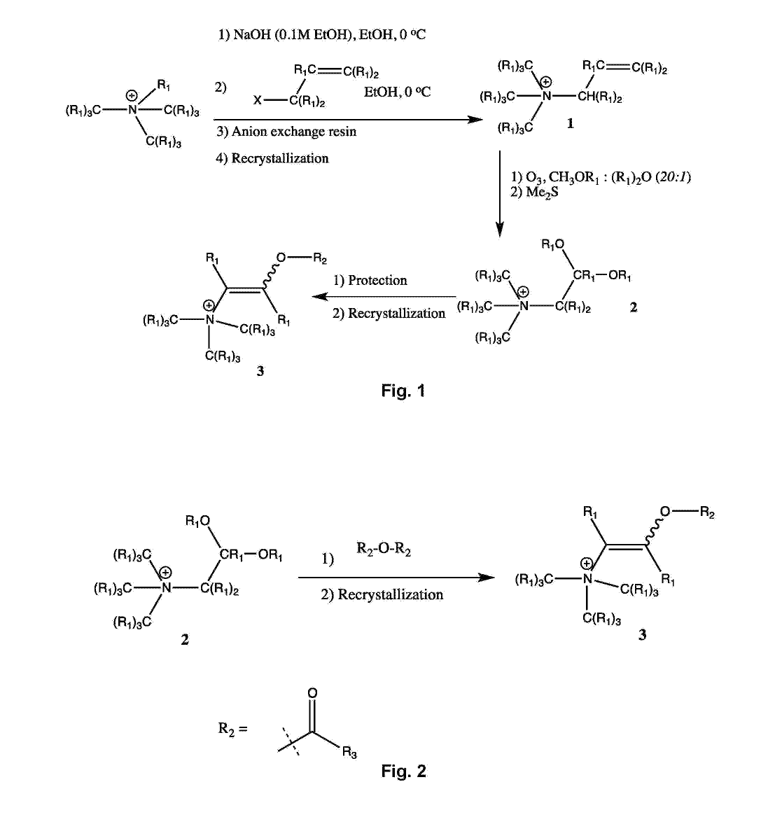

The present disclosure also provides methods of making unstaurated choline analogs. FIG. 1 shows the general synthesis of the unsaturated choline anologs disclosed herein, and includes: synthesizing allyl trimethylammonium (compound 1) from a trimethylammonium and an allyl, ozonizing the allyl trimethylammonium to form a betaine aldehyde (compound 2), and performing a protection step on the betaine aldehyde to form the unstaurated choline analog (compound 3).

In some methods, one or more of the starting reactants for synthesizing the unsaturated choline analog may include isotopically enriched atoms. For example, the nitrogen atom N of the trimethylammonium may be isotopically enriched ¹⁵N, and one or more of the protons R₁ of the trimethylammonium, and in some cases all of the protons R₁ of the trimethylammonium may be istopically enriched D. Similarly, one or more of the R₁ atoms of the allyl reactant may be isotopically enriched D. Deuterated and undeuterated ¹⁵N-trimethylammonium salts, as well as deuterated allyl reactants (such as deuterated allyl halides), are commerically available. The allyl may include any suitable leaving group X, including but not limited to a halogen, a tosyl group, a methanesulfonyl group, a trifluoromethanesolfonyl group, and trifluoroacetate, among others.

¹⁵N-trimethylammonium or deuterated ¹⁵N-trimethylammonium (e.g. perdeuterated ¹⁵N-trimethylammonium) may be basified with an alcoholic alkali and then reacted with the allyl to form the allyl trimethylammonium (compound 1). Ion exchange chromatography may be used to purify the compound, and/or to exchange the anion associated with the allyl trimethylammonium cation (e.g. to chloride instead of bromide). After recrystallization, the double bond may be cleaved by ozone (ozonolysis), and the peroxide may be reduced with dimethyl sulfide to form betaine aldehyde (or the hydroxyl form of betaine aldehyde shown in FIG. 1 as compound 2). The betaine aldehyde may be recrystallized or used in its crude uncrystallized form to perform the protection step.

As shown in FIGS. 2-5, various different reactions may be performed during the protecting step to form the various choline analogs (compound 3) of the present disclosure. The type of protecting reaction performed depends on the identity of the selected protecting group R₂.

As shown in FIG. 2, choline analogs (compound 3) having an acyl protecting group R₂ can be formed by reacting the betaine aldehyde (compound 2) with an anyhydride R₂—O—R₂, where each of the R₂ groups is an acyl —COR₃, and R₃ is H, an alkyl (e.g., —CH₃), a cycloalkyl, an alkenyl, a cycloalkenyl, an alkynyl, a cycloalkynyl, an aryl, an alkoxyl, a cyano, a thiol, an amino, or a solid support. Reaction in the neat acetic anhydride may furnish the unsaturated choline analog. The anyhydride can be a symmetrical anhydride, in which case all of the choline analogs (compound 3) produced from the reaction have the same protecting group R₂. Alternatively, the anhydride may be an unsymmetrical anhydride, such that a mixture of two different choline analogs is formed, each of the two choline analogs having a different protecting group R₂.

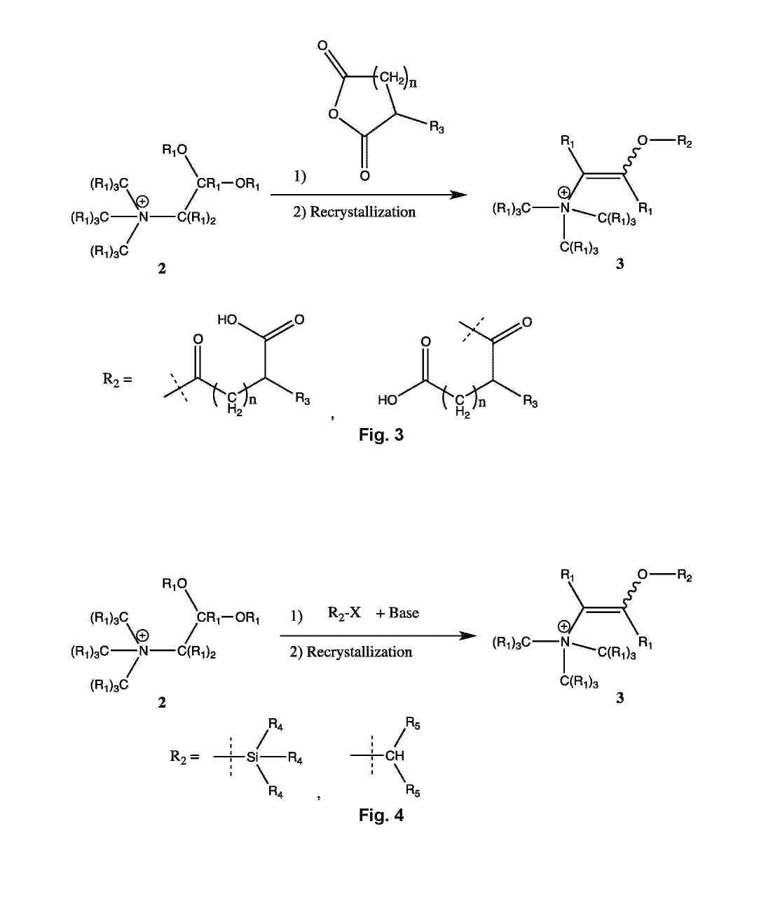

As shown in FIG. 3, the anhydride also may be a cyclic anhydride that, depending on its structure, forms two different choline analogs (compound 3), where for one of the choline analogs, R₂ is

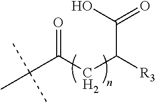

and for the other unsaturated choline analog, R₂ is

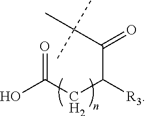

In either case, n and R₃ are the same for each of the choline analogs. Specifically, n is an integer from 1-5, and R₃ can be any of H, an alkyl (e.g., —CH₃), a cycloalkyl, an alkenyl, a cycloalkenyl, an alkynyl, a cycloalkynyl, an aryl, an alkoxyl, a cyano, a thiol, an amino, or a solid support. Cyclic anhydrides may be particularly useful for forming an unsaturated choline analog attached to a solid support (i.e., where R₃ is a solid support), because 100% of the unsaturated choline analog will be attached to the support.

Choline analogs have been formed by reacting anhydrides with betaine aldehydes (compound 2), using the specific reaction conditions described in the Examples below. It is expected that essentially any anhydride could be reacted with betaine aldehyde to form corresponding choline analogs, although for any particular reaction, the reaction conditions may need to be optimized to maximize yield. For example, reaction temperatures (between −78 C and 300 C) and purification methods (e.g., via recrystallization and/or chromatography) may need to be optimized, and solid anhydrides or anhydrides having an R₃ that is a solid support, would need to be dissolved in a co-solvent (e.g., DMSO, DMF, ethyl acetate, etc.) that would facilitate the desired reaction and would not substantially adversely react with the betaine aldehyde or the protecting-group-forming reactant.

As shown in FIG. 4, choline analogs (compound 3) also may be formed via a substitution reaction by reacting the betaine aldehyde (compound 2) with R₂—X under basic conditions, where R₂ is

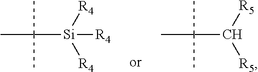

X is any leaving group (as defined above), R₄ is H, a leaving group, an acyl, an alkyl, a cycloalkyl, an alkenyl, a cycloalkenyl, an alkynyl, a cycloalkynyl, an aryl, an alkoxyl, a cyano, a thiol, an amino, or a solid support, and R₅ is H, a leaving group, an acyl, an alkyl, a cycloalkyl, an alkenyl, a cycloalkenyl, an alkynyl, a cycloalkynyl, an aryl, an alkoxyl, a cyano, a thiol, an amino, or a solid support. As with the reactions shown in FIGS. 2 and 3, for any particular reaction shown in FIG. 4, the reaction conditions may need to be optimized to maximize yield. For example, reaction temperatures (between −78 C and 300 C) and purification methods (e.g., via recrystallization and/or chromatography) may need to be optimized, and appropriate solvents selected (e.g., THF, DMF, etc.)

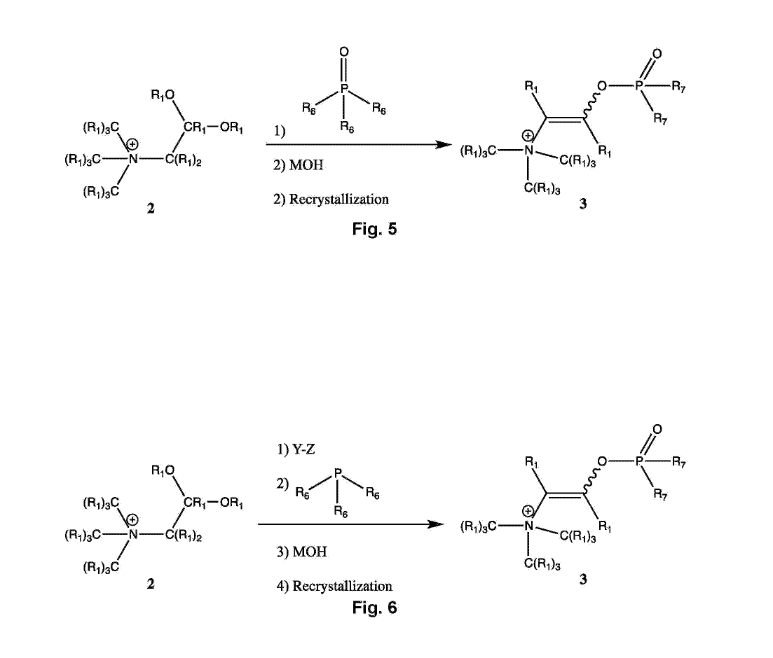

As shown in FIG. 5, choline analogs (compound 3) may be formed by reacting the betaine aldehyde (compound 2) with a phoshpine oxide PO(R₆)₃ in the presence of strong base MOH, where each R₆ is a halogen, an alkoxyl group or a hydroxyl group, and M is a cation. The unsaturated choline analog (compound 3) formed by the reaction shown in FIG. 5 has a protecting group R₂ that is

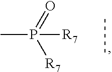

where each R₇ is an alkoxyl group or a hydroxyl group. If the protecting group R₂ is a phosphite group (—PO₃), then the unsaturated choline analog can be reduced with parahydrogen to directly form hyperpolarized phosphocholine, which may be directly administered to humans as MRI contrast agents, as discussed above.

Finally, as shown in FIG. 6, choline analogs (compound 3) may be formed by halogenating the betaine aldehyde (compound 2) with a halogenating reagent Y—Z followed by subsequent reaction with P(R₆)₃, in the presence of strong base MOH, where Y is a halogen, Z is either a halogen or any electrophilic group for promoting formation of an electrophilic halogen attached thereto, each R₆ independently is a halogen, an alkoxyl group or a hydroxyl group, and M is a cation. Exemplary halogenating agents may include, but are not limited to, N-Bromosuccinimide (NBS), Dibromoisocyanuric Acid (DBI), Poly(N-bromoacrylamide), 1-Butyl-3-methylimidazolium tribromide ([Bmim]Br3), N-Methylpyrrolidin-2-one hydrotribromide (MPHT), 2,4,4,6-tetrabromo-2,5-cyclohexadien-1-one (TBCD), N-Iodosuccinimide, Iodine monochloride, 1,3-Diodo-5,5′-dimethylhidantoin (DIN), and Iodine monobromide. Like the choline analog of FIG. 5, the unsaturated choline analog (compound 3) of FIG. 6 has a protecting group R₂ that is

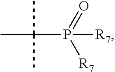

where each R₇ is an alkoxyl group or a hydroxyl group. If the protecting group R₂ is a phosphite group (—PO₃), then the unsaturated choline analog can be reduced with parahydrogen to directly form hyperpolarized phosphocholine, which may be directly administered to humans as MRI contrast agents, as discussed above.

The choline analogs (compound 3) may be used in methods of performing MRI. Specifically, as shown in FIGS. 6-10, the choline analogs (compound 3) may be reduced with parahydrogen to form hyperpolarized choline analogs that either may be directly used as a contrast agent for MRI (e.g., hyperpolarized phosphocholine) or may be further modified to form hyperpolarized choline or a hyperpolarized choline analog that may be used as a contrast agent for MRI.

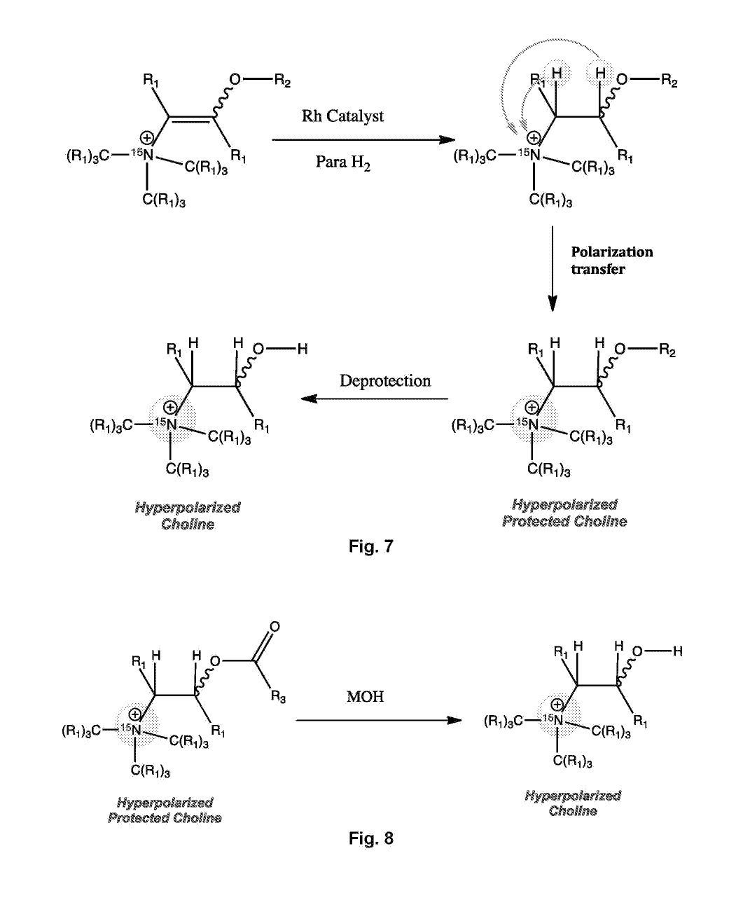

FIG. 7 is a flow chart showing the general reaction scheme for using the choline analogs of this disclosure to produce hyperpolarized choline analogs and hyperpolarized choline. An unsaturated choline analog is first reduced with parahydrogen in the presence of a rhodium catalyst, after which polarization is transferred from the parahydrogens to the ¹⁵N atom to form a hyperpolarized saturated choline analog (labeled in FIG. 6 as “Hyperpolarized Protected Choline”). As discussed above, some hyperpolarized saturated choline analogs may be useful as MRI contrast agents, whereas others may be toxic to humans and other mammals and/or otherwise may not be useful due to the nature of the protecting group. Any of the hyperpolarized choline analogs disclosed herein may subsequently be deprotected in a deprotection step to form hyperpolarized choline, where the specific nature of the deprotection step depends on the identity of the protecting group R₂.

FIG. 8 is a flow chart showing base deprotection of acyl deprotecting groups, where R₂ is COR₃, and R₃ is H, an alkyl, a cycloalkyl, an alkenyl, a cycloalkenyl, an alkynyl, a cycloalkynyl, an aryl, a cyano, or any solid support that is linked to the carbonyl of the acyl protecting group by a C—C bond.

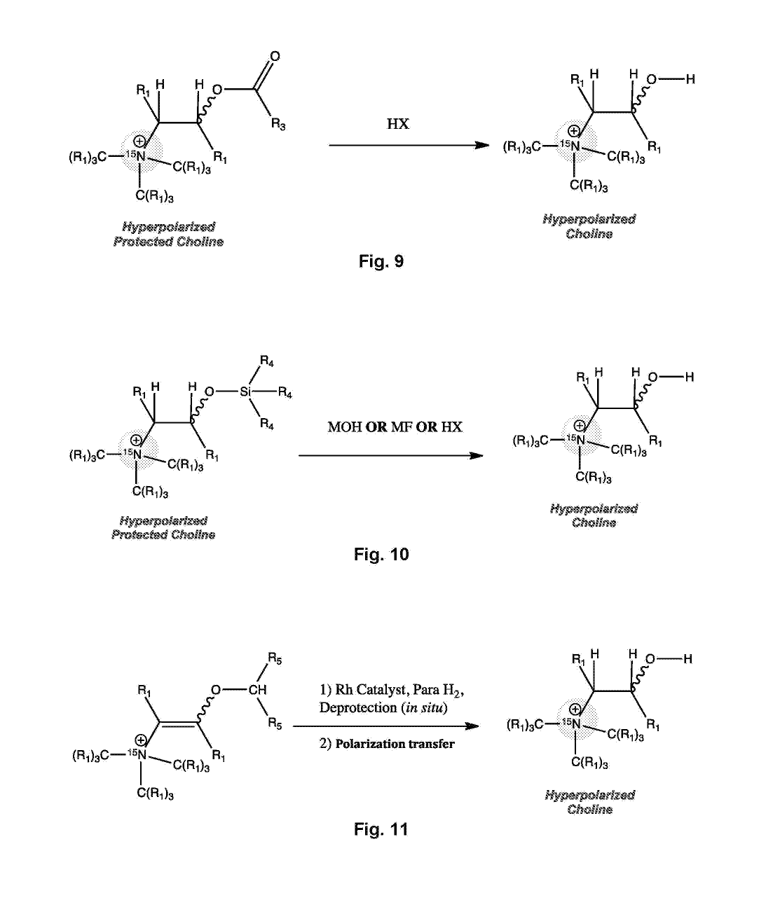

FIG. 9 is a flow chart showing acid deprotection of acyl deprotecting groups, where R₂ is COR₃, and R₃ is an alkoxyl, a thiol, an amino, or a solid support linked to the carbonyl of the acyl protecting group by a heteroatom.

FIG. 10 is a flow chart showing deprotection of silicon protecting groups with various deprotecting reagents, such as strong acids (HX, where X is any suitable leaving group), strong bases (MOH, where M is any suitable cation), or MF (e.g., NaF, Me₄NF, etc.). The specific deprotecting reagent depends on the identity of the specific silicon protecting group.

FIG. 11 is a flow chart showing deprotection of alkyl protecting groups, which depends on the structure of the protecting group. For example, where R₂ is —CH₂-aryl or —CH₂—CH═CH₂, the protecting group may be susceptible to removal during the reduction of the unsaturated choline analog with parahydrogen to directly form hyperpolarized choline, thus eliminating the need for an additional deprotection step.

### EXAMPLES

### Example 1

### Synthesis of Acetylated 1,2-Dehydrocholine Chloride

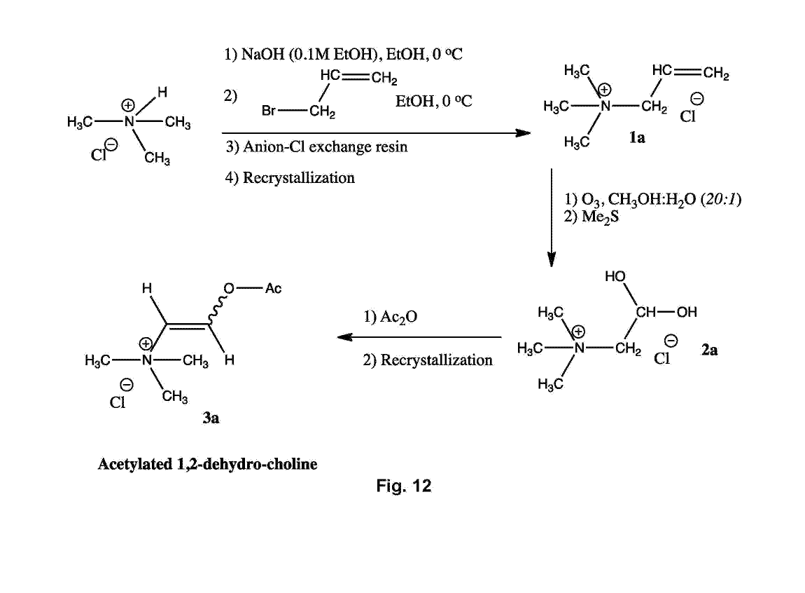

Acetylated 1,2-dehydrocholine chloride (compound 3a) was synthesized using the reaction scheme shown in FIG. 12 and described below. First, trimethylammonium chloride (5 g, 52 mmol) was dissolved in dry ethanol (200 ml) to form a first solution. The first solution was cooled to 0° C., and ice cold 1M NaOH in ethanol (52 ml, 52 mmol) was added slowly to the first solution to form a second solution. After 10 min, allyl bromide (4.5 ml, 52 mmol) was added dropwise to the second solution to form a third solution. The third solution was allowed to incubate at room temperature overnight, and was then concentrated at reduced pressure using a rotovap. The resulting solid was re-suspended in 300 ml of dry ethanol to form a fourth solution. The fourth solution was filtered in order to remove any sodium chloride/bromide salt. The resulting filtrate was run through an ion exchange column (˜400 ml of beads of IRA-400 CI, Total Volume Capacity 1.6 eq/L). The column was washed with excess ethanol, and the eluant was collected. The eluant was then concentrated in vacuo. The solid residue was recrystallize from 100 ml of ethanol by addition of 700 ml of diethyl ether, thereby furnishing 5 g (71% yield) of allyl trimethyl ammonium chloride (compound 1a).

Allyl trimethyl ammonium chloride (compound 1a) (2 g, 15 mmol) was dissolved in methanol (300 ml) and water to form a 2 ml solution. A small amount of Rose bengal was added, and the solution was cooled to −78° C. Ozone was passed through the solution until the pink color of the Rose bengal completely disappeared. Dimethyl sulfide (30 ml) was added and the reaction mixture was left at warm to room temperature overnight. The solvent was removed at reduced pressure and the resulting residue was washed with anhydrous acetone (3×50 ml). The solid residue was dried in vacuo for an additional 20 minutes, and was then recrystallized from 60 ml of anhydrous methanol followed by the addition of anhydrous diethyl ether (750 ml), thereby furnishing 1.77 g (76% yield) of betaine aldehyde (compound 2a) as a white extremely hygroscopic solid.

Betaine aldehyde (compound 2a) (1.57 g, 10.1 mmol) was suspended in 400 ml of acetic anhydride to form a reaction mixture. After 12 h, the reaction mixture was evaporated to dryness. The residue was submerged in an additional 200 ml of acetic anhydride and again evaporated to dryness. After 4 h in vacuo, the residue was partially redissolved in 250 ml of acetic anhydride and filtered. The filtrate was precipitated by the addition of dry diethyl ether (1.5 L), thereby furnishing a 1.2 g mixture of (Z) acetylated 1,2-dehydro-choline chloride (Z isomer of compound 3a), (E) acetylated 1,2-dehydro-choline chloride (E isomer of compound 3a), and di-acetylated betaine aldehyde (molar ratio of Z:E:diAc was 0.4:1:0.8), as determined by proton NMR in D₂O:

(Z) Acetylated 1,2-dehydro-choline ¹H (D₂O, 400 MHz) 7.26 (d, 1H, J=5.2), 5.76 (d, 1H, J=5.6), 3.37 (s, 9H), 2.23 (s, 3H);

(E) Acetylated 1,2-dehydro-choline ¹H (D₂O, 400 MHz) 7.89 (d, 1H, J=11.5), 6.57 (d, 1H, J=11.5), 3.30 (s, 9H), 2.14 (s, 3H);

Di-acetylated betaine aldehyde ¹H (D₂O, 400 MHz) 7.07 (t, 1H, J=4.5), 3.77 (d, 2H, J=4.5), 3.20 (s, 9H), 2.08 (s, 6H).

### Example 2

### Synthesis of Acetylated 1,2-Dehydro-Choline Bromide

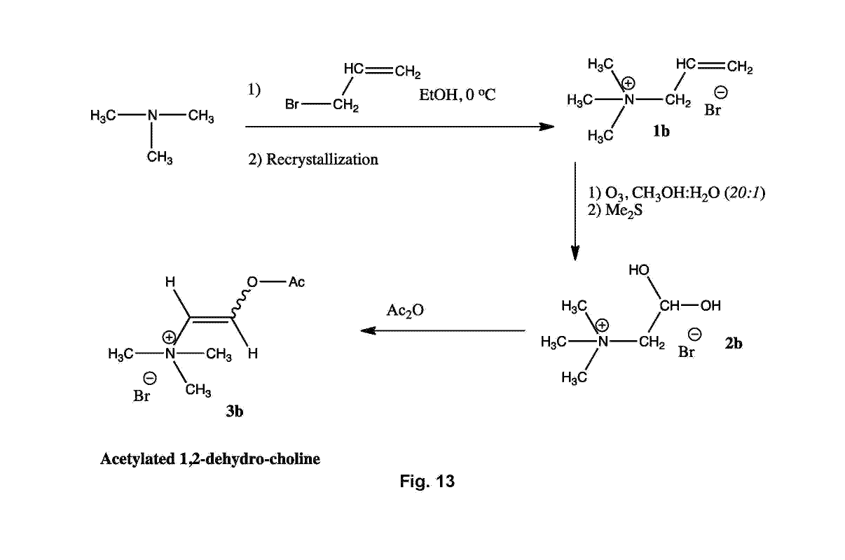

Acetylated 1,2-dehydrocholine bromide (compound 3b) was synthesized using the reaction scheme shown in FIG. 13 and described below. First, allyl trimethyl ammonium bromide (compound 1b) was synthesized using the same procedure described in Thanei-Wyss, P.; Waser, P. G., Synthesis of Reversible Inhibitors of Acetylcholinesterase (EC 3.1.1.7). Helvetica Chimica Acta 1983, 66 (7), 2198-2205, the entire disclosure of which is herein incorporated by reference for all purposes.

The allyl trimethyl ammonium bromide (compound 1b) was used to synthesize 1 g (66% yield) of betaine aldehyde (compound 2b) following the same method as was described above for the synthesis of compound 2a from compound 1a.

The betaine aldehyde (compound 2b) was used to synthesize 0.5 g (34% yield) of acetylated 1,2-dehydro-choline bromide (compound 3b) following the same method as was described above for the synthesis of compound 3a from compound 2a, except the reaction was stopped prior to completion by filtration, and no recrystallization was necessary. The sole product was a mixture of E and Z isomers of the acetylated 1,2-dehydro-choline bromide (E:Z=1:0.46).

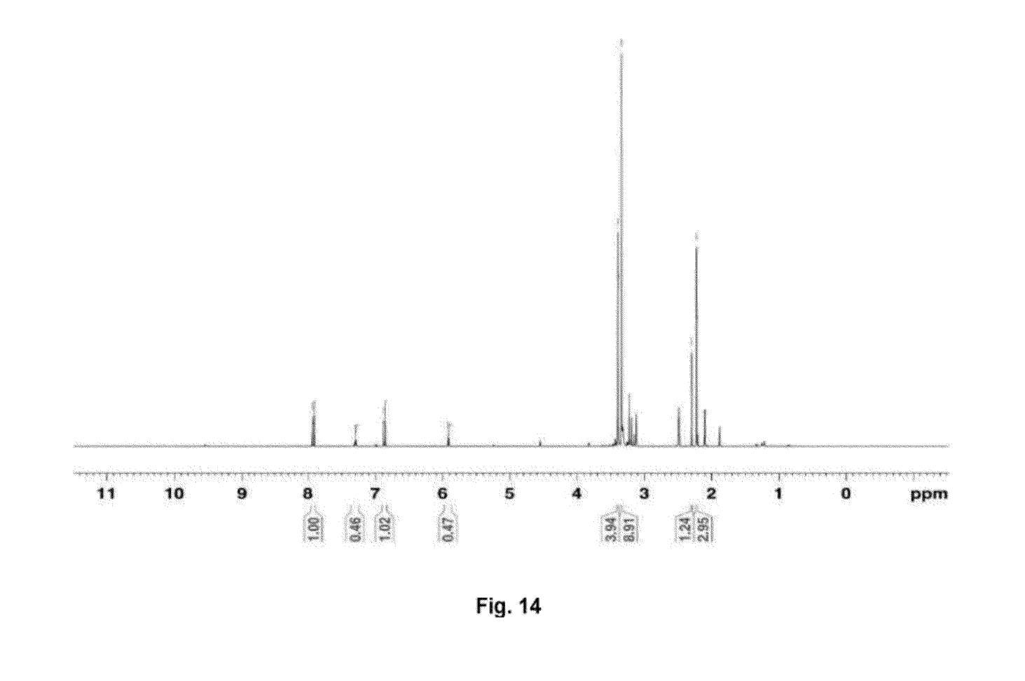

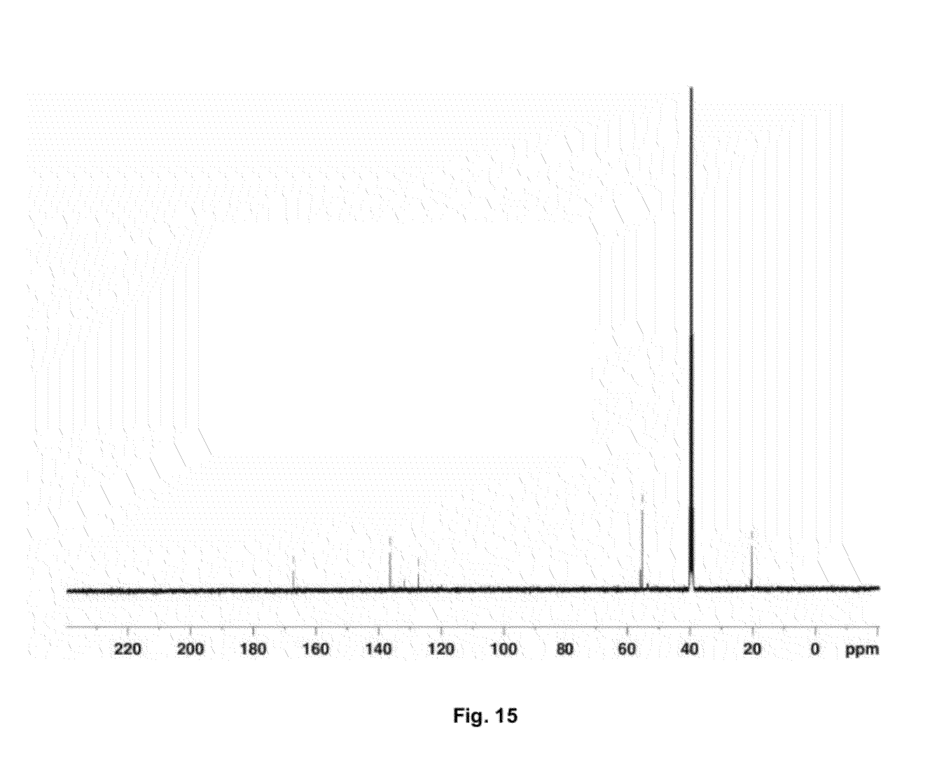

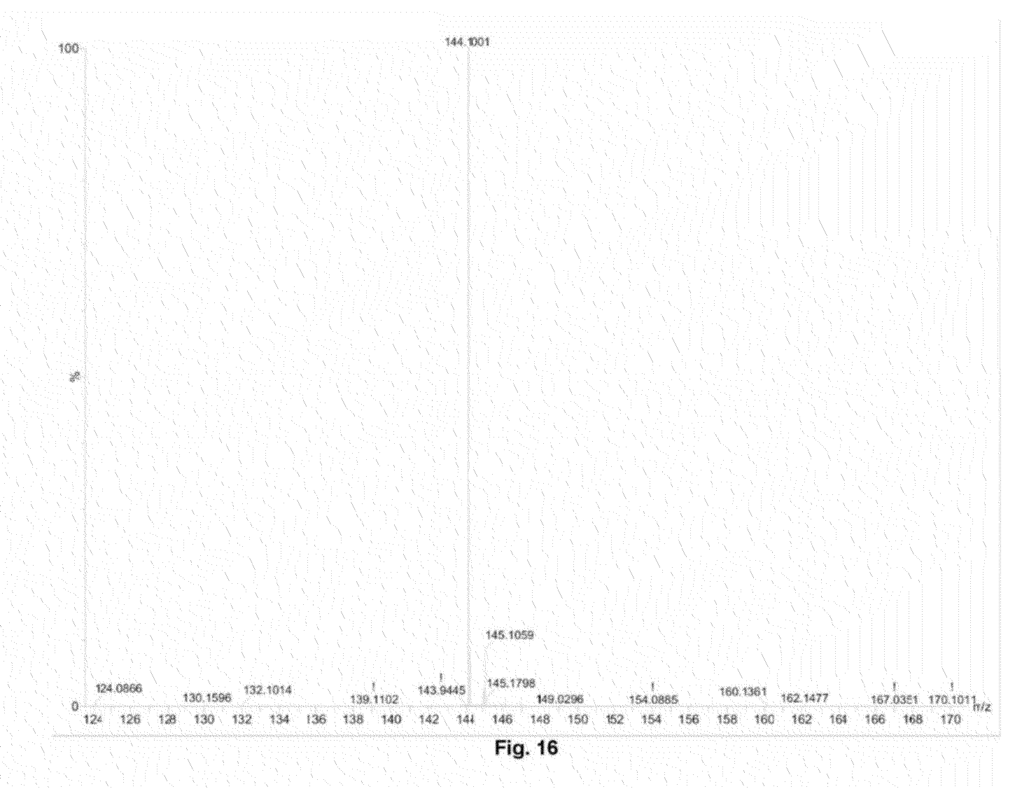

The unsaturated choline analog (compound 3b) was confirmed by assigning chemical shifts for ¹H (FIG. 14) and ¹³C (FIG. 15) NMR spectroscopy and mass spectroscopy (FIG. 16):

(E) Acetylated 1,2-dehydro-choline bromide ¹H (d-DMSO, 400 MHz) 7.93 (d, 1H, J=11.6), 6.87 (d, 1H, J=11.6), 3.34 (s, 9H), 2.23 (s, 3H); ¹³C (d-DMSO, 100 MHz) 167.2, 136.3, 127.3, 55.2, 20.2;

(E) Acetylated 1,2-dehydro-choline bromide ¹H (D₂O, 400 MHz) 7.89 (d, 1H, J=11.5), 6.57 (d, 1H, J=11.5), 3.30 (s, 9H), 2.14 (s, 3H);

(Z) Acetylated 1,2-dehydro-choline bromide ¹H (d-DMSO, 400 MHz) 7.30 (d, 1H, J=5.6), 5.92 (d, 1H, J=5.6), 3.40 (s, 9H), 2.30 (s, 3H);

(Z) Acetylated 1,2-dehydro-choline bromide ¹H (D₂O, 400 MHz) 7.26 (d, 1H, J=5.2), 5.76 (d, 1H, J=5.6), 3.37 (s, 9H), 2.23 (s, 3H);

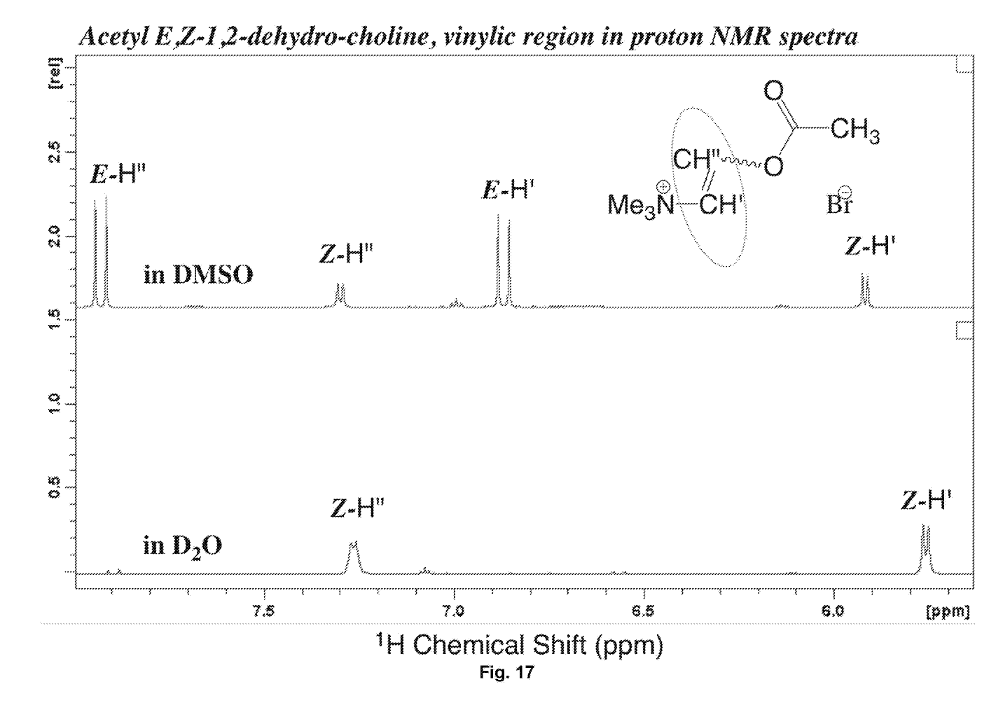

Acetyl 1,2-dehydro-cvholine also was shown to be stable for at least hours in aqueous medium. FIG. 17 shows ¹H spectra of acetyl 1,2-dehydro-choline in DMSO (top) and water (bottom). Only the Z-isomer is present in water.

### Example 3

### Synthesis of Acetylated ¹⁵N-1,2-Dehydro-Choline Bromide

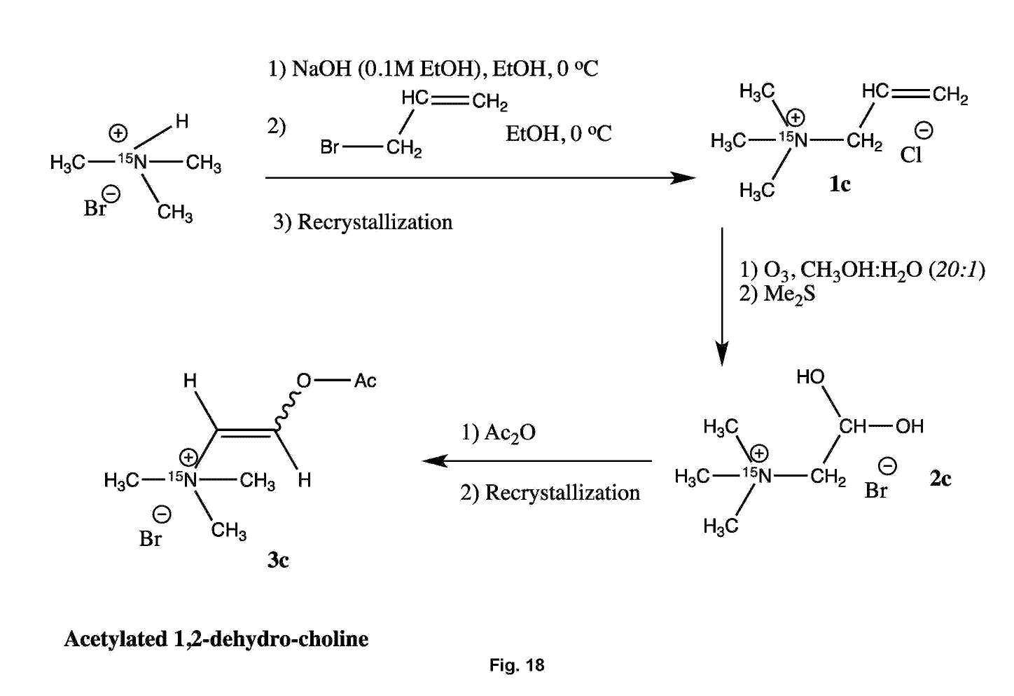

Acetylated ¹⁵N-1,2-dehydrocholine bromide (compound 3c) was synthesized using the reaction scheme shown in FIG. 18 and described below. First, 1.34 g (74% yield) of ¹⁵N-allyl trimethyl ammonium bromide (compound 1c) was synthesized from Me₃¹⁵N*HCl (0.96 g, 10 mmol) following the same method as was used to synthesize compound 1a from trimethylammonium chloride, except for the anion exchange step. The structure was confirmed by ¹H, ¹³C, ¹⁵N NMR spectroscopy and mass spectroscopy.

¹H NMR—(CD₃OD, 400 MHz) 6.10 (m, 1H), 5.74 (m, 1H), 5.71 (d of m, 1H, J=8.4), 4.00 (d, 2H, J=7.5), 3.13 (d, 9H, J=0.7);

¹³C NMR—(CD₃OD, 100 MHz) 129.5 (d, J=1.6), 126.5, 69.5 (d, J=4.3), 53.2 (d, J=5.8);

¹⁵N NMR—(CD₃OD, 40 MHz, auto calibrated) 48.87.

MS (HR) calc. for C₆H₁₄¹⁵N, 101.1091; found: 101.1092 (0.99 ppm).

1.30 g of ¹⁵N-allyl trimethyl ammonium bromide (compound 1c) was used to synthesize 0.5 g (34% yield) of ¹⁵N-betaine aldehyde (compound 2c) following the same method as was described above for the synthesis of compound 2a from compound 1a, except no purification step was performed, and the crude product containing compound 2c was used directly in the next step described below.

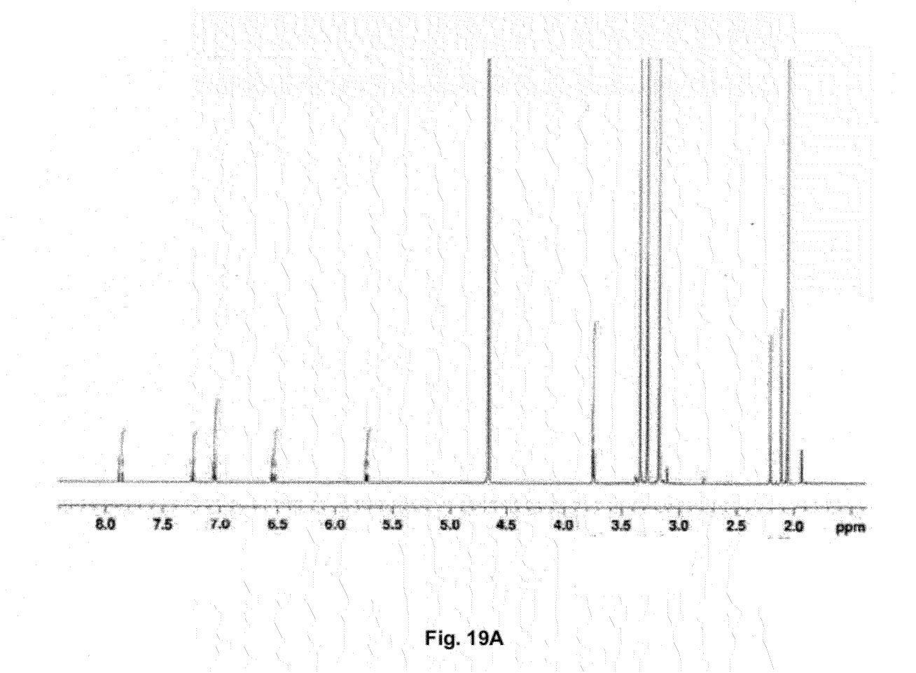

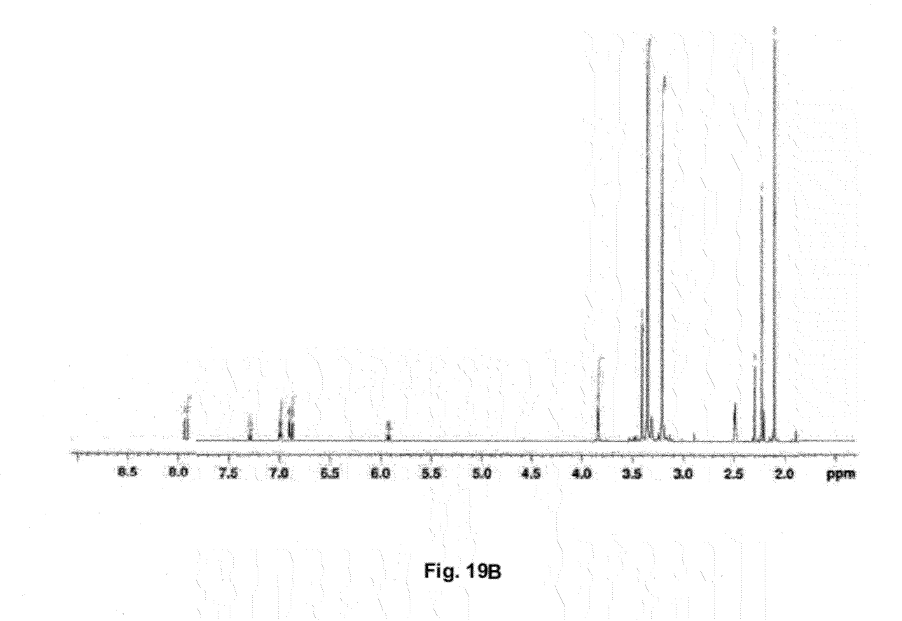

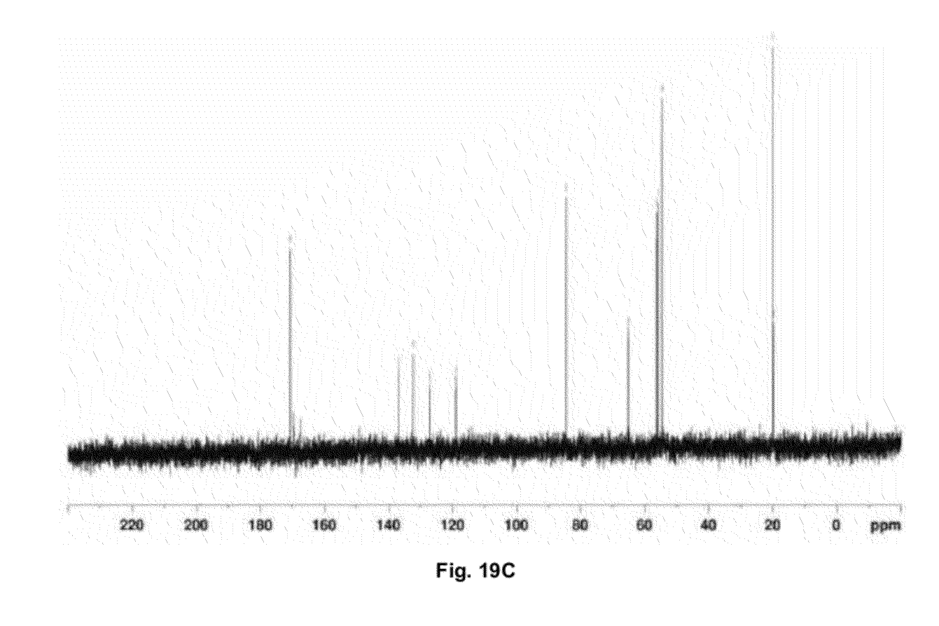

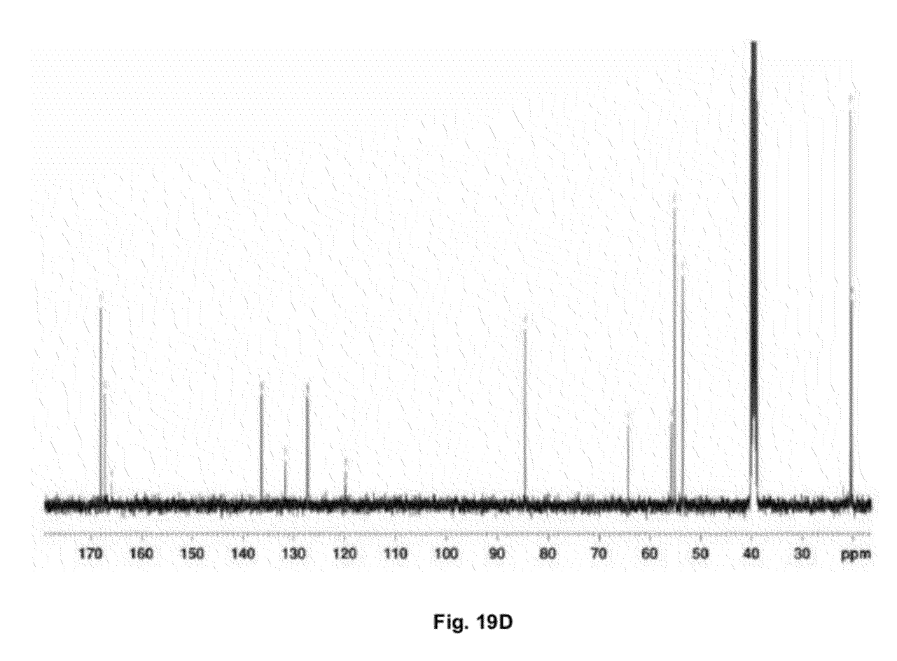

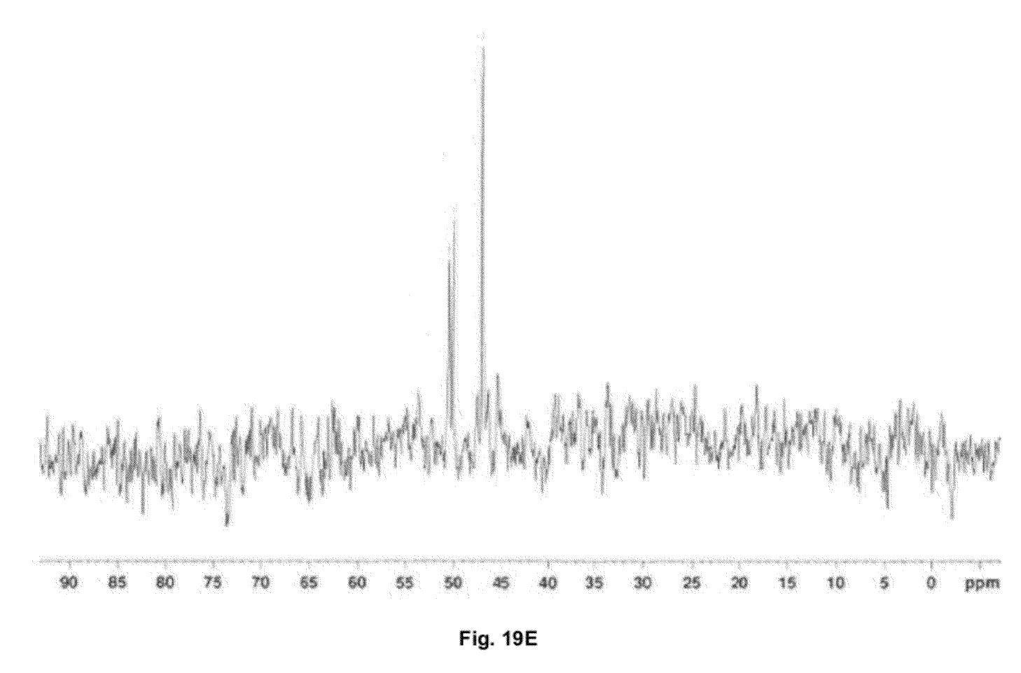

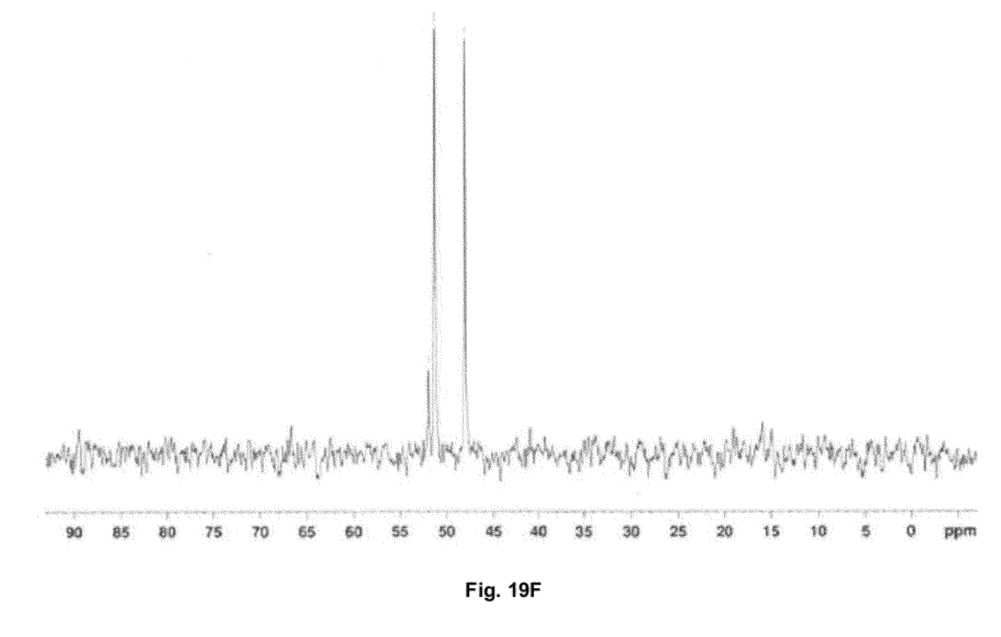

Following generally the same method as was described above for the synthesis of compound 3a from compound 2a, the ¹⁵N-betaine aldehyde (compound 2c) was used to synthesize 1.09 g of a mixture of (E) ¹⁵N-acetylated 1,2-dehydro-choline bromide (E isomer of compound 3c), (Z) ¹⁵N-acetylated 1,2-dehydro-choline bromide (Z isomer of compound 3c), and ¹⁵N-di-acetylated betaine aldehyde (molar ratio of Z:E:diAc was 0.3:1:0.9), as determined by ¹H, ¹³C and ¹⁵N NMR spectroscopy in D₂O and DMSO-d6 (FIG. 19):

(E) ¹⁵N-Acetylated 1,2-dehydro-choline bromide ¹H (d-DMSO, 400 MHz) 7.92 (d,d 1H, J_{1,2}=1.5 and 11.6), 6.89 (dd, 1H, 42=3.2 and 11.6), 3.35 (s, 9H), 2.23 (s, 3H); ¹³C (d-DMSO, 100 MHz) 167.2, 136.3 (d, J=2.4), 127.3 (d, J=9.5), 55.1 (d, J=5), 20.2; ¹⁵N (d-DMSO, 40 MHz, auto calibrated) 51.3;

(Z) ¹⁵N-Acetylated 1,2-dehydro-choline bromide ¹H (d-DMSO, 400 MHz) 7.29 (t, 1H, J=5.5), 5.92 (dd, 1H, 42=3.6 and 5.6), 3.41 (d, 9H, J=0.7), 2.30 (s, 3H); ¹⁵N (d-DMSO, 40 MHz, auto calibrated) 52.0;

¹⁵N-Diacetylated betaine aldehyde ¹H (d-DMSO, 400 MHz) 6.99 (dt, 1H, J_{1,2}=1.2 and 4.8), 3.84 (d, 2H, J=4.8), 3.21 (s, 9H), 2.10 (s, 6H); ¹⁵N (d-DMSO, 40 MHz, auto calibrated) 48.1;

### Example 4

### Synthesis of Acetylated Perdeuterated ¹⁵N-1,2-Dehydro-Choline Bromide

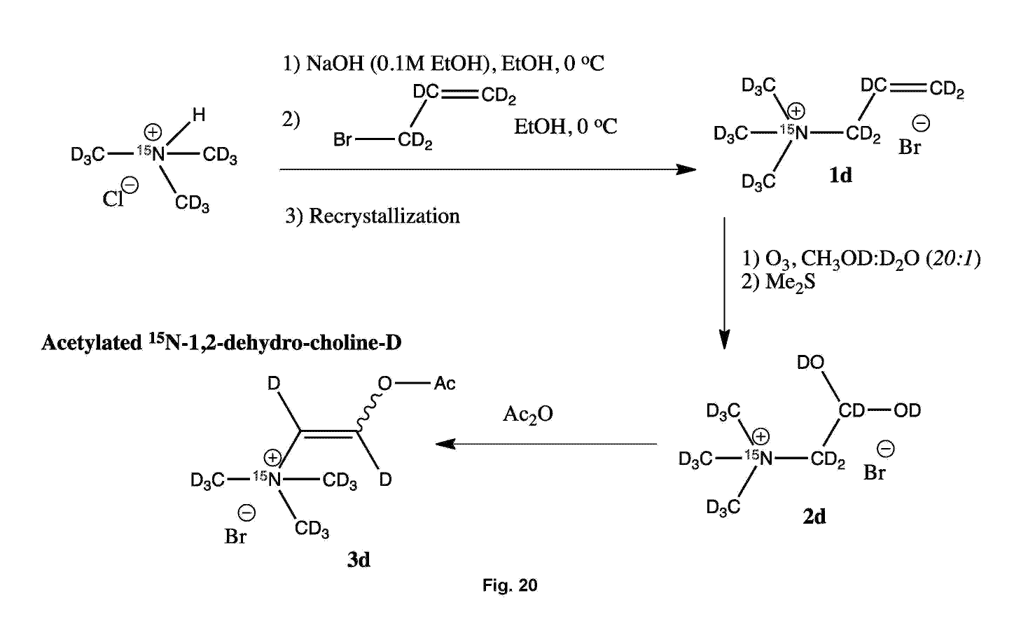

Acetylated perdeuterated ¹⁵N-1,2-dehydrocholine bromide (compound 3d) was synthesized using the reaction scheme shown in FIG. 20 and described below. First, 3.12 g (84% yield) of perdeuterated ¹⁵N-allyl trimethyl ammonium bromide (compound 1d) was synthesized from (CD₃)₃¹⁵N*HCl (2.00 g, 19 mmol) and d5-allylbromide (2.51 g, 20 mmol) following the same method as was used to synthesize compound 1a from trimethylammonium chloride and allyl bromide, except for the anion exchange step. The structure was confirmed by mass spectroscopy.

MS (HR) calc. for C₆D₁₄¹⁵N, 115.1970; found: 115.1971 (0.87 ppm).

0.95 g (4.7 mmol) of perdeuterated ¹⁵N-allyl trimethyl ammonium bromide (compound 1d) was used to synthesize perdeuterated ¹⁵N-betaine aldehyde (compound 2d) following the same method as was described above for the synthesis of compound 2a from compound 1a, except MeOD and 6d-acetone were used instead of regular methanol and acetone, no purification step was performed, and the crude product containing compound 2d was used directly in the next step described below.

The perdeuterated ¹⁵N-betaine aldehyde (compound 2d) was used to synthesize a mixture of perdeuterated ¹⁵N-acetylated 1,2-dehydro-choline bromide (compound 3d) and perdeuterated ¹⁵N-di-acetylated betaine aldehyde following the same method as was described above for the synthesis of compound 3a from compound 2a.

### REFERENCES

The following references are herein incorporated by reference in their entireties for all purposes:
1. Chekmenev, E. Y.; Hoevener, J. B.; Norton, V. A.; Harris, K. C.; Batchelder, L. S.; Bhattacharya, P.; Ross, B. D. Weitekamp, D. P. “PASADENA Hyperpolarization of Succinic Acid for MRI and NMR” J. Am. Chem. Soc. 2008, 130, 4212-4213.
2. Chekmenev, E. Y.; Chow, S.-K.; Tofan, D.; Weitekamp, D. P.; Ross, B. D.; Bhattacharya, P. “Fluorine-19 NMR Chemical Shift Probes Molecular Binding to Lipid Membranes” J. Phys. Chem. B, 2008, 112, 6285-6287. and Chekmenev, E. Y.; Norton, V. A.; Weitekamp, D. P.; Bhattacharya, P. “Hyperpolarized ¹H NMR Employing Low Gamma Nucleus for Spin Polarization Storage” J. Am. Chem. Soc. 2009, 131, 3164-3165.
3. Huang, R.; Chen, G.; Sun, M.; Gao, C., A novel composite nanofiltration (NF) membrane prepared from graft copolymer of trimethylallyl ammonium chloride onto chitosan (GCTACC)/poly(acrylonitrile) (PAN) by epichlorohydrin cross-linking. Carbohydrate Research 2006, 341 (17), 2777-2784.
4. Cabal, J.; Hampl, F.; Liska, F.; Patocka, J.; Riedl, F.; Sevcikova, K., Hydrates of Quaternary Ammonium Aldehydes as Potential Reactivators of Sarin-Inhibited Acetylcholinesterase. Collection of Czechoslovak Chemical Communications 1998, 63 (7), 1021-1030.
5. Thanei-Wyss, P.; Waser, P. G., Synthesis of Reversible Inhibitors of Acetylcholinesterase (EC 3.1.1.7). Helvetica Chimica Acta 1983, 66 (7), 2198-2205.

We claim:

1. A composition comprising a compound having the following structure: 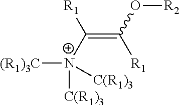
where each R₁ is independently H or is isotopically enriched D;
where R₂ is 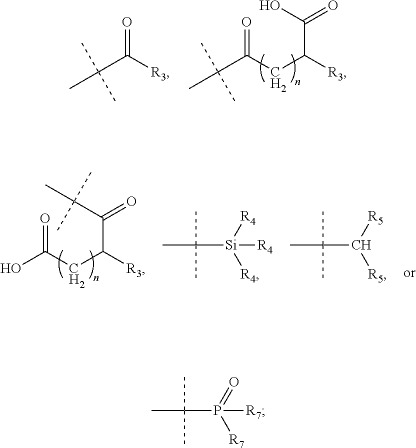
where R₃ is H, an alkyl, a cycloalkyl, an alkenyl, a cycloalkenyl, an alkynyl, a cycloalkynyl, an aryl, an alkoxyl, a cyano, a thiol, an amino, or a solid support;
where each R₄ is independently H, a leaving group, an acyl, an alkyl, a cycloalkyl, an alkenyl, a cycloalkenyl, an alkynyl, a cycloalkynyl, an aryl, an alkoxyl, a cyano, a thiol, an amino, or a solid support;
where each R₅ is independently H, a leaving group, an acyl, an alkyl, a cycloalkyl, an alkenyl, a cycloalkenyl, an alkynyl, a cycloalkynyl, an aryl, an alkoxyl, a cyano, a thiol, an amino, or a solid support;
where each R₇ is independently —OH, or an alkoxyl;
where N is ¹⁴N or isotopically enriched ¹⁵N;
where n is an integer from 1-5; and
where at least one of R₂, R₃, R₄, R₅ or R₇ comprises a negatively charged moiety,
or the analog further comprises an anion Z^{⊖} (e.g., a halide).

2. The composition of claim 1, wherein R₂ is 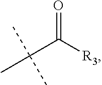
and R₃ is H, an alkyl, a cycloalkyl, an alkenyl, a cycloalkenyl, an alkynyl, a cycloalkynyl, an aryl or a solid support.

3. The composition of claim 1, wherein R₂ is 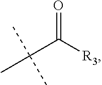
and R₃ is —CH₃.

4. The composition of claim 1, wherein R₂ is 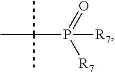
and each R₇ is —OH.

5. The composition of claim 1, wherein the compound is in the form of a chloride salt.

6. The composition of claim 5, wherein the compound is:

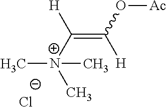

7. The composition of claim 1, wherein the compound is in the form of a bromide salt.

8. The composition of claim 7, wherein the compound is:

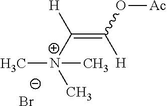

9. The composition of claim 1, wherein N is isotopically enriched ¹⁵N.

10. The composition of claim 1, wherein at least one R₁ is isotopically enriched D.

11. The composition of claim 10, wherein each R₁ is isotopically enriched D.

12. A method of making a choline analog, comprising:
performing a protection step on a betaine aldehyde to form the choline analog.

13. The method of claim 12, further comprising:
synthesizing allyl trimethylammonium from a trimethylammonium and an allyl; and
ozonizing the allyl trimethylammonium to form the betaine aldehyde.

14. The method of claim 13, wherein the nitrogen atom of the trimethylammonium salt is isotopically enriched ¹⁵N.

15. The method of claim 13, wherein at least one of the hydrogen atoms of the trimethylammonium salt is istopically enriched D.

16. The method of claim 13, wherein at least one of the hydrogen atoms of the allyl is isotopically enriched D.

17. The method of claim 12, wherein at least some of the betaine aldehyde exists in the following form:

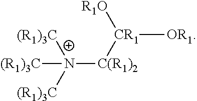

18. The method of claim 12, wherein the protection step includes reacting the betaine aldehyde with an anhydride.

19. The method of claim 18, wherein the anhydride is a cyclic anhydride.

20. The method of claim 19, wherein the cyclic anhydride comprises a substituent group that includes a solid support.

21. The method of claim 12, wherein the protection step includes reacting the betaine aldehyde with a protecting group that includes silicon.

22. The method of claim 12, wherein the protection step includes reacting the betaine aldehyde with a phosphine oxide under hydrolytic conditions to form the following compound:

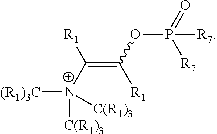

23. The method of claim 12, wherein the protection step includes first reacting the betaine aldehyde with a halogenating reagent to form a halogenated intermediate, and then reacting the halogenated intermediate with P(R₆)₃ under hydrolytic conditions, where each R₆ independently is a halogen, an alkoxyl group or a hydroxyl group, thereby forming the following compound:

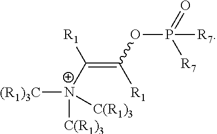

24. A method of performing MRI, comprising:
reducing the compound of claim 1 with parahydrogen to form a hyperpolarized choline analog.

25. The method of claim 24, wherein the hyperpolarized choline analog is hyperpolarized phosphocholine.

26. The method of claim 25, further comprising administering the hyperpolarized choline analog to a patient to detect cancer.

27. The method of claim 24, wherein the hyperpolarized choline analog is a hyperpolarized protected choline precursor, and the method further comprises deprotecting the hyperpolarized protected choline precursor to form hyperpolarized choline.

28. The method of claim 27, further comprising administering the hyperpolarized choline to a patient to detect cancer.
